(19) United States
(12) Patent Application Publication
(10) Pub. No.: US 2023/0149170 A1
(43) Pub. Date: May 18, 2023

(54) DEVICES, SYSTEMS, AND METHODS FOR POSITIONING A LEAFLET CLIP

(71) Applicant: BOSTON SCIENTIFIC SCIMED, INC., Maple Grove, MN (US)

(72) Inventors: Troy Anthony Giese, Blaine, MN (US); Joel T. Eggert, Plymouth, MN (US); Nicholas Barron, Columbia Heights, MN (US); Matthew P. Jones, Shoreview, MN (US); Kristen Elizabeth Ott, White Bear Lake, MN (US)

(73) Assignee: BOSTON SCIENTIFIC SCIMED, INC., MAPLE GROVE, MN (US)

(21) Appl. No.: 17/899,469

(22) Filed: Aug. 30, 2022

Related U.S. Application Data
(60) Provisional application No. 63/279,473, filed on Nov. 15, 2021.

(51) Int. Cl. A61F 2/24

(52) U.S. Cl. CPC A61F 2/2466; A61F 2/2454; A61F 2250/0096; A61F 2240/001; A61F 2220/0008

(57) ABSTRACT

Implantable devices formed of materials which are not readily imageable are delivered and deployed with a deployment/delivery device having one or more sensors generating a signal indicating a condition of the implantable device relative to the deployment site. For instance, the signal indicates at least one or more of the following: purchase of the implantable device with tissue, level of purchase of the implantable device with tissue, the position of the implantable device relative to the deployment site, seating of tissue with respect to the implantable device, extent of contact of the implantable device with tissue, or further information about the implantable device and/or the delivery/deployment device. As such, the implantable device need not be imaged to determine the relationship of the implantable device relative to the deployment site.

### CROSS-REFERENCE TO RELATED APPLICATIONS

This application claims the benefit of priority of U.S. Provisional Application No. 63/279,473, filed Nov. 15, 2021, the entire disclosure of which is hereby incorporated by reference herein for all purposes.

### FIELD

The present disclosure relates generally to the field of implantable medical devices. In particular, the present disclosure relates to medical devices, systems, and methods for cardiac treatment. More particularly, the present disclosure relates to medical devices, systems, and methods for coupling a leaflet clip to a heart valve, such as to deliver and implant artificial chordae tendineae in a heart.

### BACKGROUND

Heart disease, including atrioventricular heart valve malfunctions, impedes patient cardiac output, which reduces patient quality of life and lifespan. The proper flow of blood through the heart is regulated, inter alia, by heart valves, including atrioventricular heart valves, which include soft tissue leaflets which cyclically open and close to allow blood to flow through in one direction. Healthy leaflets prevent blood flow in the opposite direction (regurgitation). Chordae tendineae, extending from the leaflets to the papillary muscles, support the proper functioning of the leaflets, such as by distributing load to the papillary muscles during systolic closure, and by preventing the leaflet from flailing into the atrium. As heart disease progresses, the chordae tendineae that connect the papillary muscle of the ventricle to a valve leaflet may stretch inelastically and may rupture. Various defects or failure or other improper functioning of the chordae tendineae, such as elongation, rupture, thickening, retraction, calcification, inelastic stretching, or other changes in elasticity, etc., may result in improper closure (e.g., sealing) of the heart valve and/or a flailing leaflet that may no longer have the capacity to form a valving seal for normal heart function. Abnormal blood flow regurgitation from the ventricle to the atrium may develop, preventing an adequate supply of blood to be delivered through the cardiovascular systems.

Various leaflet clips have been developed to repair damaged or otherwise impaired leaflets. Such leaflet clips may be deployed using transcatheter techniques without requiring open heart surgery. Transcatheter or otherwise noninvasive procedures (not requiring open and invasive surgery) generally require simultaneous visualization of both the targeted anatomy and the implant tools (delivery and deployment tools, as well as the implant itself). Unfortunately, current visualization tools are incapable of identifying both tissue as well as inorganic materials from which implant tools are made (e.g., metals, polymers, ceramics, etc.). Ultrasound easily identifies tissue, but struggles with many foreign materials, especially metallic components commonly implemented in medical devices and associated mechanisms and systems. As such, the devices and systems tend to cast shadows obstructing the view of targeted tissues and often the devices and mechanisms as well. Fluoroscopy is well suited for viewing dense materials, but is generally unsuitable for visualizing many soft tissues (such as heart leaflets).

Solutions for verifying the purchase of an implanted medical device, such as a heart valve leaflet clip, with tissue and/or proper implantation of a medical device would be welcome.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

In accordance with various principles of the present disclosure, a delivery/deployment device is configured to deliver and/or deploy an implantable device at a deployment site and includes one or more sensors along a portion of the delivery/deployment device configured to engage the implantable device. In some aspects, the one or more sensors are positioned and configured to generate a signal so that the delivery/deployment device and the implantable device need not be imaged to determine the relationship of the implantable device relative to the deployment site.

In some embodiments, the delivery/deployment device includes a seat in which an implantable device is positionable. In some embodiments, the one or more sensors extend along at least one side of the seat.

In some embodiments, the delivery/deployment system further including an indicator coupled with the one or more sensors and configured to generate a signal indicating a condition of the delivery/deployment device and/or an implantable device associated therewith relative to the deployment site.

In some embodiments, the one or more sensors are formed by a vapor deposition process.

In some embodiments, the one or more sensors are impedance sensors capable of distinguishing body tissue.

In some embodiments, the delivery/deployment device is a clip spreader configured to operatively engage a clip having a first clip arm and a second clip arm to shift the clip between an open configuration in which the first clip arm and the second clip arm are spaced apart from each other to allow tissue to be positioned therebetween, and a closed configuration in which the first clip arm and the second clip arm are adjacent to each other to clamp tissue therebetween.

In accordance with various principles of the present disclosure, a system for delivering and/or deploying an implantable device to a deployment site in a body includes a clip, a clip spreader, and one or more sensors associated with the clip. In some aspects, the clip has a first clip arm and a second clip arm, the clip being movable between a closed configuration in which the first clip arm and the second clip arm are adjacent to each other, and an open configuration in which the first clip arm and the second clip arm are spaced apart from each other to allow tissue to be positioned therebetween. In some aspects, the clip spreader is configured to operatively engage the clip to shift the clip between the open configuration and the closed configuration. In some aspects, the one or more sensors are associated with the clip to generate a signal indicating a condition of the clip relative to the deployment site.

In some embodiments, the one or more sensors are provided on the clip spreader to be positioned adjacent the clip engaged by the clip spreader. In some embodiments, the clip spreader includes a seat in which one of the arms of the clip is engaged, the one or more sensors being arranged along at least one side of the seat to extend along at least one side of the one of the arms of the clip engaged within the seat. In some embodiments, clip spreader includes a first spreader arm configured to operatively engage the first clip arm, and a second spreader arm configured to operatively engage the second clip arm; the seat is formed in at least the second spreader arm; movement of the first spreader arm away from the second spreader arm moves the clip to the open configuration; and the one or more sensors are arranged along at least the second spreader arm along at least one side of the seat formed therein.

In some embodiments, the signal indicates at least one or more of the following: purchase of the clip with tissue at the deployment site, level of purchase of the clip with tissue at the deployment site, the position of the clip relative to the deployment site, seating of tissue at the deployment site between the arms of the clip, extent of contact of the clip with tissue at the deployment site, or further information about the clip and/or the clip spreader.

In some embodiments, the one or more sensors generate an audible or visual signal indicating a condition of the clip relative to the deployment site.

In some embodiments, the one or more sensors are formed by a vapor deposition process.

In some embodiments, the one or more sensors are impedance sensors capable of distinguishing body tissue.

In accordance with various principles of the present disclosure, a method of delivering and/or deploying an implantable device to a deployment site in a body includes delivering an implantable device to a deployment site using a delivery/deployment device, where one or more sensors are associated with the implantable device; and moving the one or more sensors towards the deployment site to cause the one or more sensors to generate a signal indicating a condition of the implantable device relative to the deployment site.

In some embodiments, the method further includes deploying the implantable device based on the signal generated by the one or more sensors verifying purchase with tissue.

In some embodiments, the one or more sensors generate a signal upon contact with tissue at the deployment site.

In some embodiments, the implantable device is a leaflet clip having a first clip arm and a second clip arm, and the method further includes moving the leaflet clip into an open configuration in which the first clip arm and the second clip arm are spaced apart from each other to allow a heart valve leaflet to be positioned therebetween; and moving the first clip arm and the second clip arm towards each other based on the signal generated by the one or more sensors verifying purchase with tissue.

In some embodiments, the implantable device is a leaflet clip having a first clip arm and a second clip arm, and the method further includes moving the leaflet clip into an open configuration in which the first clip arm and the second clip arm are spaced apart from each other to allow a heart valve leaflet to be positioned therebetween; moving the first clip arm and the second clip arm towards each other and verifying the desired position of the leaflet clip with respect to the heart valve has been reached; and causing the leaflet clip to clamp onto the heart valve leaflet positioned between the first clip arm and the second clip arm.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of a leaflet clip system formed in accordance with various principles of the present disclosure positioned to deploy a clip with respect to a schematic representation of a heart valve leaflet.

FIG. 2 is a perspective view of an example of an embodiment of a leaflet clip system formed in accordance with various principles of the present disclosure positioned to deploy a clip with respect to a schematic representation of a heart valve leaflet.

FIG. 3 is a perspective view showing the clip spreader of a leaflet clip system as in FIG. 2 opening an example of an embodiment of a leaflet clip.

FIG. 4A illustrates a perspective view of an example of an embodiment of a leaflet clip spreader and leaflet clip formed in accordance with various principles of the present disclosure positioned to deploy a clip in a first position with respect to a schematic representation of a heart valve leaflet.

FIG. 4B illustrates a perspective view similar to that of FIG. 4B, but with the clip in a second position with respect to a schematic representation of a heart valve leaflet further advanced on the leaflet clip than the first position.

FIG. 5 is a cross-sectional view along line V-V of a leaflet clip system as in FIG. 2.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and “distal” refers to the direction or location furthest from the user, such as when using the device (e.g., introducing the device into a patient, or during implantation, positioning, or delivery), and/or closest to a delivery device and/or a treatment site. “Longitudinal” means extending along the longer or larger dimension of an element. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore.

Repositioning, repair, and/or replacement of one or more leaflets of a valve and/or chordae tendinea may be desirable to treat heart disease. The devices, systems, and methods of the present disclosure may be used alone or together with other devices, systems, and methods to treat heart disease. Examples of devices, systems, and methods with which embodiments of the present disclosure may be implemented include, but are not limited to, those described in U.S. Patent Application Publication US2021/0007847, titled Devices, Systems, And Methods For Clamping A Leaflet Of A Heart Valve, and published on Jan. 14, 2021; U.S. Patent Application Publication US2021/0000597, titled Devices, Systems, And Methods For Adjustably Tensioning An Artificial Chordae Tendineae Between A Leaflet And A Papillary Muscle Or Heart Wall, and published on Jan. 7, 2021; U.S. Patent Application Publication US2021/0000599, titled Devices, Systems, And Methods For Artificial Chordae Tendineae, and published on Jan. 7, 2021; U.S. Patent Application Publication US2021/0000598, titled Devices, Systems, And Methods For Anchoring An Artificial Chordae Tendineae To A Papillary Muscle Or Heart Wall, and published on Jan. 7, 2021; U.S. Patent Application Publication 2022/0096235, titled Devices, Systems, And Methods For Adjustably Tensioning Artificial Chordae Tendineae In A Heart, and published Mar. 31, 2022; and U.S. patent application Ser. No. ______ [ATTORNEY DOCKET 2001.2700100], filed on even date herewith, filed on even date herewith, each of which is herein incorporated by reference in its entirety and for all purposes. Examples of devices described therein may be modified to incorporate embodiments or one or more features of the present disclosure.

Devices, systems, and methods described herein facilitate fixation of one or more devices to tissue, such as to tissue of a heart valve (e.g., a heart valve leaflet) by clamping a device to the tissue, such as with the use of a clamp or clip. It will be appreciated that terms such as clip, clamp, clasp, couple, engage, attach, deploy on, grasp, hold, etc. (including other grammatical forms thereof) may be used interchangeably herein, such as with reference to the interaction of the clip and tissue engaged by the clip, without intent to limit. Repositioning, repair, and/or replacement of one or more leaflets of a valve and/or chordae tendinea may include one or more devices to be fixed to one or more leaflets of a heart valve, such as with a leaflet clip. Examples of embodiments of devices and systems described herein may provide a fixed point for other devices, systems, or tools to grab or attach to in order to manipulate a leaflet of a valve and/or deliver devices attached to the leaflet. For instance, intravenous or transcatheter chordae tendineae replacement requires locating and attaching an artificial chordae tendineae (such as an ePTFE suture) to the dysfunctional leaflet edge, such as with a leaflet clip. Leaflet clips may be formed from a metal (e.g., nitinol or surgical grade stainless steel), which is difficult to visualize while within the body while also visualizing the tissue (e.g., leaflet edge) to which the clip is to be clamped.

In accordance with various principles of the present disclosure, one or more sensors are associated with an implantable device and system such as to convey information about the implantable device, such as with respect to the deployment site. The information may include, without limitation, purchase (e.g., engagement) of the implantable device with the deployment site, indication of the level (e.g., degree) of purchase of the implantable device with the deployment site, the position of the implantable device relative to the deployment site, the seating of tissue at the deployment site with respect to the implantable device, spatial extent (e.g., length or surface area) of contact of the implantable device with the deployment site (e.g., tissue at the deployment site), further information about the device or system, etc. It will be appreciated that terms such as deployment site, treatment site, implantation site, etc. may be used interchangeably herein without intent to limit. The implantable device may be a device for securing another implantable device in place, such as a clip (e.g., a leaflet clip). The implantable device may include a device for securing another implantable device in place as well as one or more implantable devices operatively associated therewith. The one or more sensors may be associated with the implantable device, such as to convey information about the engagement of the implantable device with tissue at the deployment site. The one or more sensors may be associated with one or more components of the implantable device. Additionally or alternatively, the one or more sensors may be associated with one or more components of a delivery/deployment device and/or a delivery/deployment system for delivering and/or deploying the implantable device, such as to convey information about the implantable device and/or the delivery/deployment device and/or the delivery/deployment system. It will be appreciated that references herein to delivery and/or deployment are intended to include delivery, or deployment, or both. It will further be appreciated that deployment may include securing in addition to placing of a device. The delivery/deployment device may be configured to hold and/or carry the implantable device for delivery to the deployment site, and/or to facilitate deployment of the implantable device. For instance, the delivery/deployment device may be configured to manipulate the implantable device for positioning and deploying (e.g., securing, implanting, anchoring, etc.) with respect to the deployment site. It will be appreciated that terms such as manipulate (and other grammatical forms thereof) may be used interchangeably herein with such terms (and other grammatical forms thereof) as actuate, control, maneuver, move, operate, shift, transition, drive, advance, retract, rotate, translate, etc., without intent to limit.

Various implantable devices usable with devices, systems, and methods disclosed herein, and or delivery/deployment devices and/or systems disclosed herein are formed of (or components thereof are formed of) a metal or other material which is not readily visible or visualized or imaged or otherwise discernible with typical or current imaging systems used for transluminal delivery of an implantable device. For instance, metal components typically are visible under fluoroscopy, but tissue is not. And, tissue is generally visible under ultrasound imaging, but implantable devices generally are not sufficiently clear to the medical professional (the implantable devices often give off a lot of artifact, obscuring details necessary for performance of the desired procedures). In accordance with various principles of the present disclosure, sensors are associated with the implantable device and/or the delivery/deployment device or system to provide information not readily discernible with typical or current imaging technology so that the delivery/deployment device and/or the implantable device need not be imaged or visualized to determine the relationship (e.g., spatial, degree of contact, etc.). In some embodiments, the one or more sensors are contact sensors. For instance, the one or more sensors may provide a direct indication of the level of tissue engagement at the time of purchase. The one or more sensors relay a signal to the medical professionals using the system to implant the device (e.g., a surgical team) when in contact with tissue, indicating that the device is engaging the tissue sufficiently or in a desired position or location. Such information is conveyed to the medical professionals (e.g., surgical team, including any automated system being utilized) delivering/deploying the device in any of a variety of manners (e.g., visual or audible indication, such as on a delivery system or separate associated device) to facilitate placement, implantation, etc., of the device. For instance, such information may be used to guide placement of the device prior to engagement with (e.g., grasping of) tissue and/or prior to deployment of the device, and/or to determine if repositioning is warranted. More than one sensor may be provided to indicate the spatial extent (e.g., length or surface area) of contact with tissue, the position of the device relative to the tissue (e.g., clip position relative to a leaflet), the degree of purchase of the device with tissue, further information with regard to the implantable device, etc. In some embodiments, multiple sensors can be used, such as to generate various information, such as to indicate both tissue contact as well as the position of the device relative to the tissue. Electronics associated with the sensors can sense resistance, impedance, capacitance, etc. of tissue in contact with the sensors, such as between the sensors (e.g., between electrodes of the sensors). In some embodiments, one or more electrodes (e.g., an electrical conductor used to make contact with a nonmetallic part of a circuit) are components of the sensor (device, module, machine, or subsystem whose purpose is to detect events or changes). Optimum electrode spacing and the most appropriate sensing method (e.g., to optimize the signal/noise ratio for detecting the targeted tissue) may be determined based on the tissue to which the medical device is to be associated, For instance, optimum electrode spacing, material, and excitation method may differ based on the targeted material and environment. In some embodiments, the one or more sensors transmit signals generating visual (e.g., an illuminated signal indicating engagement generally, or providing further detailed visual information) and/or audible indicators (e.g., a sound indicating engagement generally, or providing further information), such as on a delivery system or separate associated device. Other forms and manners of providing information with regard to the device, system, method, etc., are within the scope of the present disclosure, the broad concepts not being so limited.

It will be appreciated that the sensors may be formed of conductive materials. Additional conductive components, such as leads, traces, connection pathways, solder pads, etc., may be associated with the sensors, such as to convey information to the medical professionals and/or to supply the sensors with power. The conductive materials may be selected from any of a variety of materials such as, without limitation, titanium, niobium, gold, nickel-copper, silver, tin, platinum, palladium, iridium, tantalum, tungsten, zirconium, copper, nickel, etc.

In an embodiment in which the device is a leaflet clip to be clamped onto a heart valve leaflet, it generally is desirable for the leaflet clip not only to have good purchase with the heart valve leaflet, but also for the heart valve leaflet to be positioned between arms of the leaflet clip in an appropriate configuration. For instance, it generally is desirable for the heart valve leaflet to be fully seated within a leaflet clip, such as with an edge of the heart valve leaflet extended to the back of the leaflet clip (where the arms of the leaflet clip which clamp onto the leaflet, meet). Additionally or alternatively, it generally is desirable for the heart valve leaflet to be clamped by the leaflet clip without being bunched or rolled on top of itself, or otherwise folded or contorted (which may affect functioning of the heart valve leaflet). It will be appreciated that typical leaflet clips formed of resilient and/or shape memory materials such as stainless steel or Nitinol generally cannot be visualized effectively with the imaging technique used to visualize tissue effectively. The provision of one or more sensors in association with the leaflet clip allows virtual visualization of the leaflet clip within the body without actual visualization. In some embodiments, a clip spreader (which may be also known as a grasper) is utilized to deliver and/or deploy the leaflet clip. The one or more sensors may be provided on the clip spreader adjacent the leaflet clip so that contact of the sensors with tissue indicates corresponding contact of the leaflet clip with tissue.

In the examples disclosed herein, the implantable device is a leaflet clip, and the sensors are provided on the surface of a leaflet clip spreader configured to grasp and spread the arms of the leaflet clip to receive tissue (e.g., a heart valve leaflet) therebetween. As such, various principles of the present disclosure are described herein with reference to embodiments of a leaflet clip and associated devices, systems, and mechanisms. However, it will be appreciated that the principles of the present disclosure may be applied more broadly to other devices, systems, methods, etc., configured to engage body tissue. The devices, systems, and methods described herein provide robust solutions for patient safety, particularly with regard to implantable devices. Accordingly, it will be appreciated that devices, systems, and methods described herein may be used with any of the devices, systems, methods, etc., disclosed in the above-referenced applications incorporated herein, or may be used with other devices, systems, methods, etc., such as those described herein or otherwise.

Various embodiments of devices, systems, and methods for positioning an implantable device will now be described with reference to examples illustrated in the accompanying drawings. Reference in this specification to “one embodiment,” “an embodiment,” “some embodiments”, “other embodiments”, etc. indicates that one or more particular features, structures, and/or characteristics in accordance with principles of the present disclosure may be included in connection with the embodiment. However, such references do not necessarily mean that all embodiments include the particular features, structures, and/or characteristics, or that an embodiment includes all features, structures, and/or characteristics. Some embodiments may include one or more such features, structures, and/or characteristics, in various combinations thereof. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. When particular features, structures, and/or characteristics are described in connection with one embodiment, it should be understood that such features, structures, and/or characteristics may also be used in connection with other embodiments whether or not explicitly described, unless clearly stated to the contrary. It should further be understood that such features, structures, and/or characteristics may be used or present singly or in various combinations with one another to create alternative embodiments which are considered part of the present disclosure, as it would be too cumbersome to describe all of the numerous possible combinations and subcombinations of features, structures, and/or characteristics. Moreover, various features, structures, and/or characteristics are described which may be exhibited by some embodiments and not by others. Similarly, various features, structures, and/or characteristics or requirements are described which may be features, structures, and/or characteristics or requirements for some embodiments but may not be features, structures, and/or characteristics or requirements for other embodiments. Therefore, the present disclosure is not limited to only the embodiments specifically described herein.

Turning now to the drawings, it will be appreciated that common features are identified by common reference elements and, for the sake of brevity and convenience, and without intent to limit, the descriptions of the common features are generally not repeated. For purposes of clarity, not all components having the same reference number are numbered.

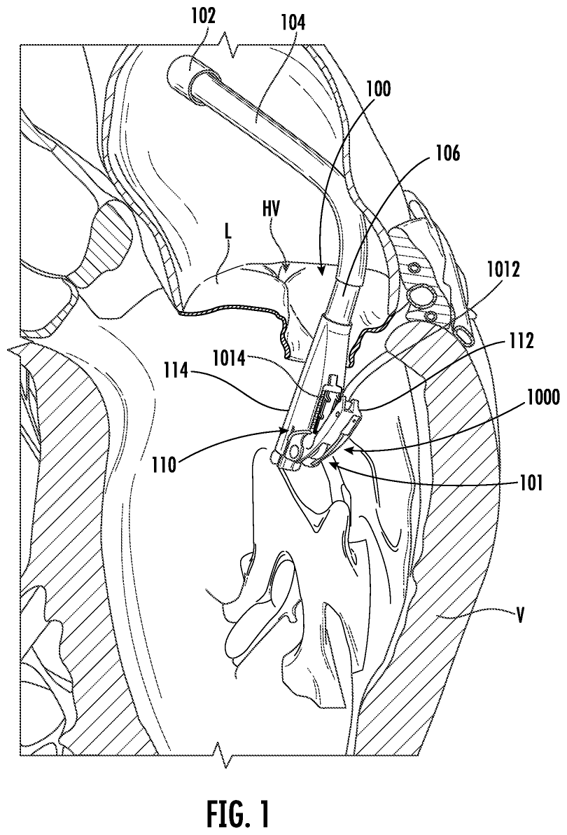

An example of an embodiment of a system and device to which various principles of the present disclosure may be applied is a delivery and deployment system 100, such as illustrated in FIG. 1. The illustrated example of an embodiment of a delivery and deployment system 100 is a leaflet clip delivery and deployment system 100 which may be delivered to the deployment site by a delivery catheter 102 which may be guided within a delivery guide sheath 104. The delivery catheter 102 and the delivery guide sheath 104 may be flexible tubular elements (e.g., catheter, sheath, shaft, tube, etc.) steerable through tortuous pathways through the body to allow for transluminal (e.g., transcatheter, in contrast with open surgery) delivery of a leaflet clip 1000. The delivery guide sheath 104 may be introduced into the body with a dilator through the femoral artery to cross through the septal wall into the ventricle. The delivery catheter 102 may be steerable (such as formed with articulations) to be positioned substantially centered above the mitral valve. The implantable device may be carried by a shaft 106 extendable out of the delivery catheter 102 or retractable into the delivery catheter 102 (e.g., telescoping in or out of the delivery catheter 102) to be brought into position relative (e.g., closer) to a heart valve leaflet L without moving out of the alignment with the heart valve leaflet L established by the delivery catheter 102.

The leaflet clip delivery and deployment system 100 is illustrated in FIG. 1 as delivering a leaflet clip 1000 to a deployment or treatment site within a heart, and in position to deploy the leaflet clip 1000 on a heart valve leaflet L. The leaflet clip delivery and deployment system 100 may include a clip spreader 110 configured and positioned to facilitate positioning and delivery of the leaflet clip 1000 to the deployment site (e.g., a heart valve leaflet L). For instance, the leaflet clip 1000 may be coupled to the clip spreader 110 for delivery and deployment with the clip spreader 110 to a deployment site. It will be appreciated that terms such as coupled with, engaged with, operatively associated with, carried by, etc. (and other grammatical forms thereof) may be used interchangeably herein without intent to limit. The clip spreader 110 may also be configured and positioned with respect to the leaflet clip delivery and deployment system 100 to carry the leaflet clip 1000 to the deployment site for deployment. In some embodiments, the clip spreader 110 may be provided on an end of a clip spreader shaft 106 such as described above.

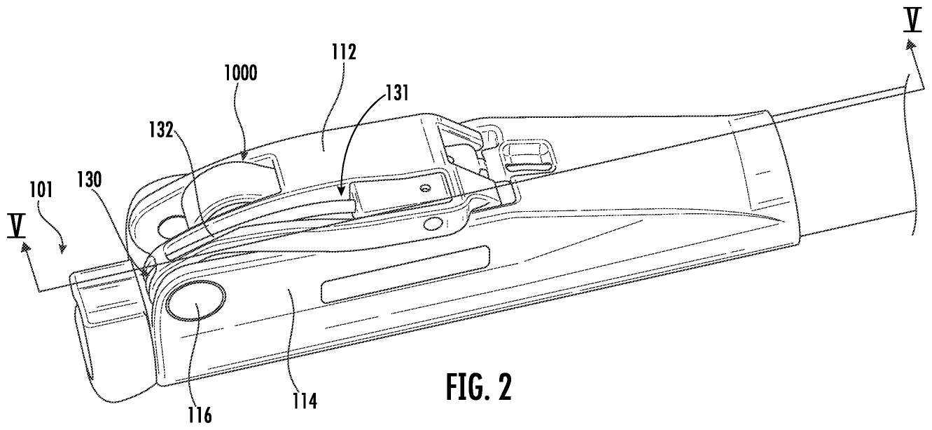

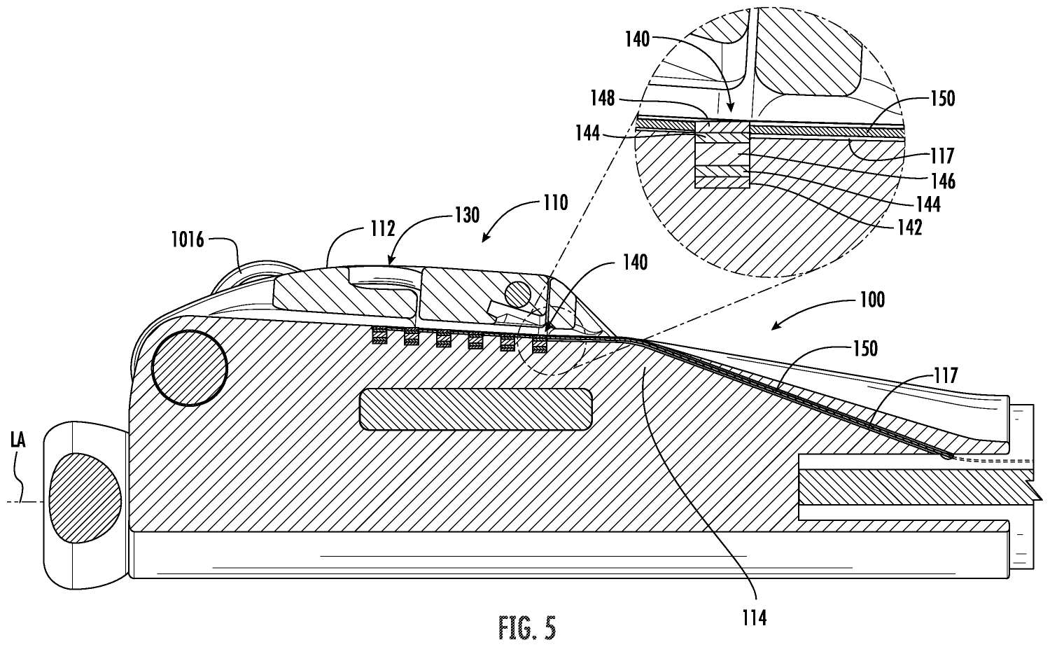

The leaflet clip 1000 may be any known leaflet clip, or a leaflet clip such as disclosed and described herein or in any of the patents or patent applications incorporated herein. Generally, the leaflet clip delivery and deployment system 100 delivers a leaflet clip 1000 having at least one ventricular clip arm 1012 configured to be positioned on a side of the heart valve leaflet L facing the heart ventricle, and at least one atrial clip arm 1014 configured to be positioned on a side of the heart valve leaflet L facing the heart atrium. In various examples of embodiments of leaflet clips 1000 disclosed herein, when the ventricular clip arm 1012 and the atrial clip arm 1014 of the leaflet clip 1000 are spaced apart from each other in an open configuration, tissue such as a heart valve leaflet L, may be positioned therebetween. When the ventricular clip arm 1012 and the atrial clip arm 1014 are in a closed configuration closer to each other (adjacent to, and optionally contacting each other if another element is not positioned therebetween), the heart valve leaflet L may be clamped between the ventricular clip arm 1012 and the atrial clip arm 1014, as described in further detail below. The ventricular clip arm 1012 and the atrial clip arm 1014 may be biased towards each other in a closed clamping configuration, and movable about a flex zone 1016 (see, e.g., FIG. 2 and FIG. 5) into an open configuration to accept body tissue such as a heart valve leaflet L between the arms 1012, 1014. In some embodiments, the leaflet clip arms 1012, 1014 engage each other when the leaflet clip 1000 is in a closed configuration such that the leaflet clip arms 1012, 1014 clamp onto tissue positioned therebetween. However, it will be appreciated that the disclosed leaflet clip delivery and deployment system 100 and associated leaflet clip 1000 need not be so limited.

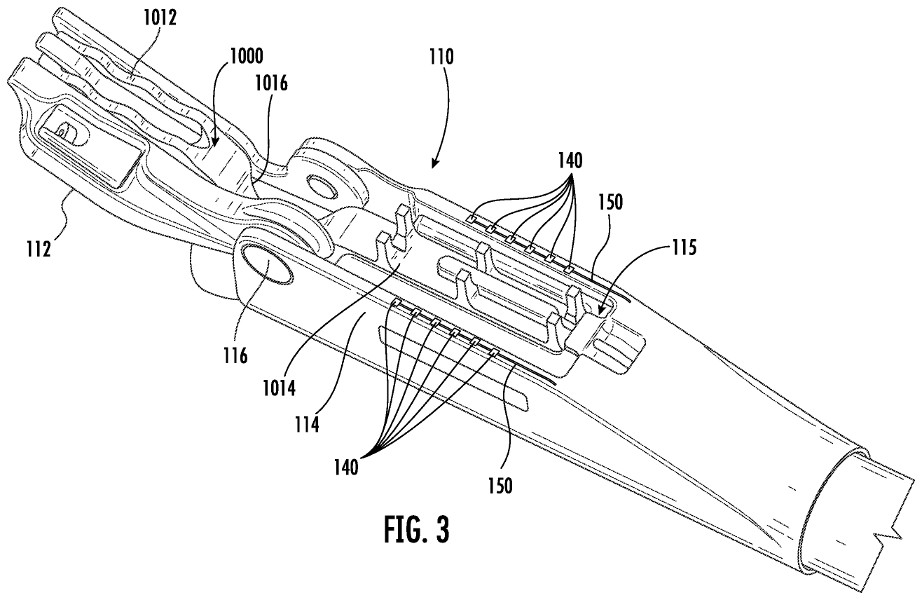

In accordance with various principles of the present disclosure, the example of an embodiment of a leaflet clip delivery and deployment system 100 illustrated in FIG. 1 includes a clip spreader 110, as noted above, which may be used to manipulate the ventricular clip arm 1012 and the atrial clip arm 1014 with respect to each other to engage a heart valve leaflet L (e.g., therebetween). It will be appreciated that the term manipulated (and other grammatical forms thereof) may be used interchangeably herein with terms such as actuated, moved, controlled, maneuvered, etc., without intent to limit. In the example of an embodiment of a clip spreader 110 illustrated in further detail in FIG. 2 and FIG. 3, the clip spreader 110 includes a ventricular spreader arm 112 configured to engage the ventricular clip arm 1012 of the leaflet clip 1000, and an atrial spreader arm 114 configured to engage the atrial clip arm 1014 of the leaflet clip 1000. The ventricular spreader arm 112 and the atrial spreader arm 114 may be pivotably coupled together, such as about a hinge or pivot 116. The pivot 116 may be a pivot point or pin, or other structure allowing relative movement of the ventricular spreader arm 112 and the atrial spreader arm 114 such as known to those of ordinary skill in the art, the details of which are not critical to the present disclosure. Movement of the ventricular spreader arm 112 with respect to the atrial spreader arm 114 causes movement of the ventricular clip arm 1012 with respect to the atrial clip arm 1014. For instance, the clip spreader arms 112, 114 may be moved apart to move the leaflet clip arms 1012, 1014 apart to allow tissue to be positioned therebetween. The clip spreader arms 112, 114 may then be moved together (e.g., returned to a closed position in which the clip spreader arms 112, 114 are close together) to move the leaflet clip arms 1012, 1014 together to grasp the tissue therebetween.

In embodiments in which the ventricular clip arm 1012 and the atrial clip arm 1014 of the leaflet clip 1000 are biased towards each other, coupling of the clip spreader 110 with the leaflet clip 1000 may allow the leaflet clip 1000 to maintain the ventricular spreader arm 112 and the atrial spreader arm 114 of the clip spreader 110 in a generally closed configuration (such as illustrated in FIG. 2) until actuated into an open configuration (such as illustrated in FIG. 3). The ventricular spreader arm 112 may be actuated, such as with an actuator 130, to shift the arms 112, 114 of the clip spreader 110 relative to each other, such as by moving the ventricular spreader arm 112 with respect to the atrial spreader arm 114, to thereby move the ventricular clip arm 1012 and the atrial clip arm 1014 of the leaflet clip 1000 (coupled, respectively, to the ventricular spreader arm 112 and the atrial spreader arm 114). In the illustrated example of an embodiment, the actuator 130 includes an actuator cable 132 having a distal end 131 coupled to the ventricular spreader arm 112, and a proximal end (not shown) accessible (e.g., outside the patient) by a medical professional to actuate the actuator 130. The actuator cable 132 may be a metal, polymer, or other suitable biocompatible material capable of withstanding the forces necessary to actuate the clip spreader 110 as well as able to be navigated through a tortuous pathway within the patient's body. In some embodiments, the actuator cable 132 is in the form of a Bowden cable.

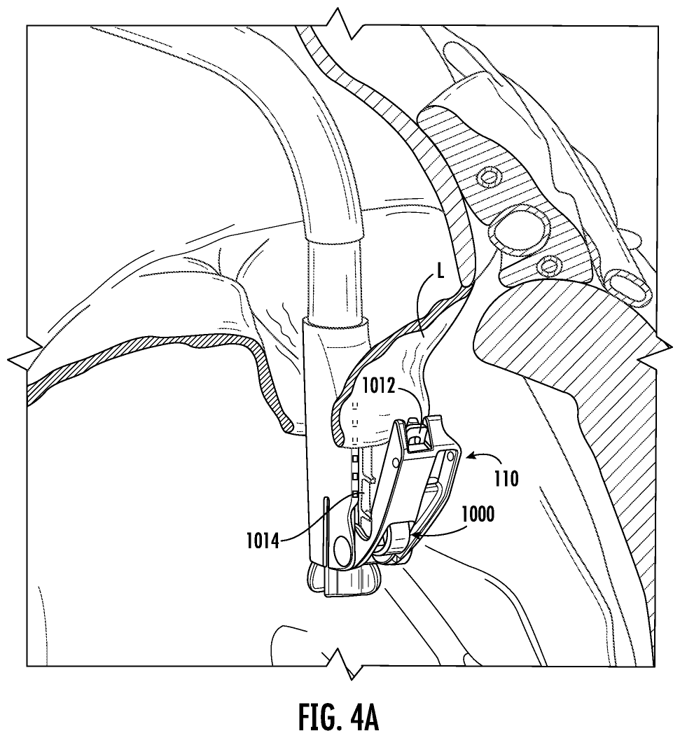

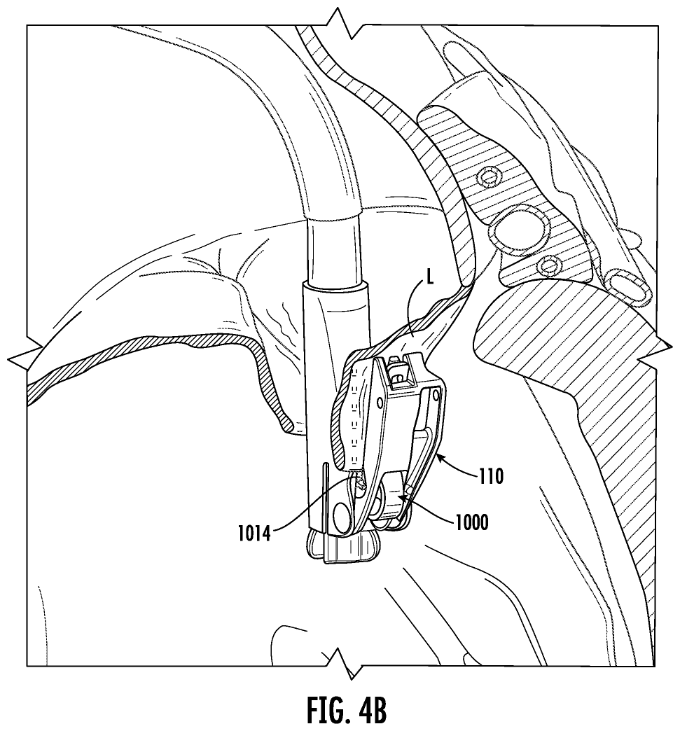

In accordance with various principles of the present disclosure, the clip spreader 110 is configured to move at least one arm of the leaflet clip 1000 with respect to another arm of the leaflet clip 1000 to shift the leaflet clip 1000 into an open configuration to allow tissue, such as a heart valve leaflet L, to be positioned between the leaflet clip arms 1012, 1014, as illustrated in FIG. 4A. In the example of an embodiment illustrated in FIG. 1, FIG. 2, FIG. 3, FIG. 4A, and FIG. 4B, the clip spreader 110 is configured to move the ventricular clip arm 1012 away from the atrial clip arm 1014, however other configurations are within the scope of the present disclosure. The leaflet clip delivery and deployment system 100 may be extended through a heart valve HV so that the distal end 101 thereof, with the leaflet clip 1000, is within the ventricle V, such as illustrated in FIG. 1. The leaflet clip 1000 may be opened, such as by actuating the clip spreader 110 to move the ventricular clip arm 1012 away from the atrial clip arm 1014, to allow the heart valve leaflet L to enter the space within the leaflet clip 1000 between the arms 1012, 1014, such as illustrated in FIG. 4A. Optionally, the leaflet clip delivery and deployment system 100 may be moved proximally towards the heart valve leaflet L to capture the heart valve leaflet L between the arms 1012, 1014. The ventricular clip arm 1012 may then be allowed to return to a neutral position closer to the atrial clip arm 1014, grasping the heart valve leaflet L therebetween, such as illustrated in FIG. 4B.

In accordance with various principles of the present disclosure, to facilitate proper placement and/or sufficient purchase with tissue (e.g., to secure another implantable device to the tissue, such as an artificial chordae tendineae and associated tissue anchor), one or more sensors 140 may be associated with the leaflet clip delivery and deployment system 100. For instance the one or more sensors 140 may be associated with the clip spreader 110 and/or the leaflet clip 1000. The sensors 140 may be contact sensors (e.g., including electrodes generating a signal upon contact with tissue), or any other sensor or device capable of indicating the position and/or engagement of the leaflet clip 1000 with tissue.

In the example of an embodiment illustrated in FIG. 3, sensors 140 are provided on the clip spreader 110 adjacent to the leaflet clip 1000. In some embodiments, as may be appreciated with reference to FIG. 3, the clip spreader 110 may include a seat 115 in which the leaflet clip 1000 (e.g., an arm of the leaflet clip 1000 such as the atrial clip arm 1014) may be positioned. Sensors 140 may be positioned on a shoulder 118 of the clip spreader 110 alongside the seat 115 and thus in proximity to the leaflet clip 1000 (e.g., a portion thereof, such as an arm thereof). For instance, in the example of an embodiment illustrated in FIG. 3, one or more sensors 140 are positioned on a shoulder 118 on the atrial spreader arm 114 alongside a seat 115 in which the atrial clip arm 1014 of the leaflet clip 1000 is positioned. It will be appreciated that one or more sensors 140 may be positioned on one or both sides of the leaflet clip 1000, such as to allow the sensors 140 to indicate that the heart valve leaflet L is fully positioned within the leaflet clip 1000. As illustrated in the example of an embodiment of FIG. 3, the sensors 140 may be arranged in a generally linear array, each extending along the longitudinal axis LA (of the leaflet clip delivery and deployment system 100 and/or the clip spreader 110) with a first array on a first side of the leaflet clip 1000 and a second array on a second side of the leaflet clip 1000 (spaced apart from the first array in a direction generally transverse to the longitudinal axis LA). In some embodiments, depending on the configuration and type of sensors used, in some embodiments, sensors 140 are associated with both leaflet clip arms 1012, 1014, such as by being positioned along both clip spreader arms 1012, 1014. It will be appreciated that in such embodiments, sensors 140 may be associated with one or both sides of each of the leaflet clip arms 1012, 1014.

Various suitable sensors are available to generate useful and usable signals indicating whether the leaflet clip 1000 is in position or in contact or otherwise ready for deployment and/or properly deployed, such as illustrated in FIG. 4A, FIG. 4B, and FIG. 5. In accordance with various principles of the present disclosure, one or more sensors 140 are provided which can identify the tissue being contacted. The sensors 140 may include contact or proximity sensors or other types of sensors known to those of ordinary skill in the art. In some embodiments, the sensor is an impedance sensor which measures impedance of the material being contacted or otherwise in the proximity or sensing range or region of the sensor. Such impedance-based sensors may be used in technology used in electrophysiology devices which map the heart, such as based on electrical activity of the heart. The sensors 140 may be configured to constantly/continuously take measurements of impedance of materials contacted by (or otherwise in a sensing range of) the sensors 140. Optionally, the sampling may be performed at a rate selected to assure accuracy of placement without unnecessary delays. Based on the measurements of the different impedances of the different materials contacted by the sensors 140, signals may be generated to indicate whether the leaflet clip 1000 is contacting tissue. For instance, the sensors 140 and associated circuitry may be configured to distinguish body tissue from other materials and/or to distinguish different types of tissue (e.g., muscle, fat, etc.) based on the impedance measured upon contact with such tissue.

Generally, the sensors 140 generally are or at least include electrically conductive components. It is also common for components of the leaflet clip delivery and deployment system 100 (such as the clip spreader 110 and/or the leaflet clip 1000) to be formed at least in part of electrically conductive material, such as nitinol or medical grade stainless steel (e.g., 316 Stainless Steel). As those of ordinary skill in the art would appreciate, it generally would be desirable to electrically insulate the sensors 140 from electrically conductive components of the leaflet clip delivery and deployment system 100. In accordance with various principles of the present disclosure, a nonconductive material 142 (e.g., an insulative material) may be positioned between the sensors 140 and the component of the leaflet clip delivery and deployment system 100 on which the sensors 140 are to be mounted (e.g., an arm of the clip spreader 110), such as illustrated in FIG. 5. The nonconductive material may be ceramic, polymer (e.g., hard plastic, polyamides, parylene, metal oxides, nitrides, etc.), etc. or combinations thereof. More particularly, any of a variety of ceramics, such as, without limitation, aluminum-based ceramics, zirconium-based ceramics, silicon carbide, boron carbide, boron nitride, silicon nitride, aluminum nitride, etc., may be used. The nonconductive material may be applied to one or more surfaces of one or more components of the leaflet clip delivery and deployment system 100 in any desired manner including, without limitation, adhesion, interference fit (e.g., fitting in a groove, pocket, recess, etc.), physical vapor deposition, electroless plating, electrolytic plating, brazing, or other bonding method. It will be appreciated that a standard circuit board (e.g., a small board with electrodes adhered to the deployment head) may be utilized. In some embodiments, the nonconductive material is applied with a physical vapor deposition, such as sputter-coating, thermal evaporation, arc spraying, etc. Limited surface areas of components of the leaflet clip delivery and deployment system 100, or the entire surface of one or more components of the leaflet clip delivery and deployment system 100 may be covered with a nonconductive material.

The sensors 140 may be mounted on the nonconductive material 142 directly or indirectly, such as via a bonding or tie layer 144 (herein after “tie layer” for the sake of convenience and without intent to limit) as illustrated in FIG. 5. The tie layer 144 may be formed of any material known for such purposes, such as, without limitation, titanium, molybdenum, chromium, etc. The tie layer 144 may be applied in any of a variety of manners such as described above with respect to the nonconductive material 142. In some embodiments, physical vapor deposition is advantageously used. Traces can be applied by conductive printing or a preformed electrode can be bonded to the insulator by a brazing process and/or by adhesive.

As noted above, in some embodiments, the sensors 140 are configured to sense and generate an impedance measurement, and thus are electrically conductive. The sensors 140 may be formed of a conductive material. The conductive portion or layer 146 (such terms being used interchangeably herein without intent to limit) of the sensors 140 provide a primary contact with tissue. The conductive portion 146 of the sensors 140 may be in the form of a conductive layer 146 coupled (e.g., bonded) to an implantable device and/or deliver/deployment system such as via a tie layer 144 as described above. The conductive layer 146, which is preferably also biocompatible (such as, without limitation, niobium, silver, titanium, tungsten, tantalum, etc., as well as nitrides or oxides) may be formed/deposited in any of a variety of manners such as described above with respect to the nonconductive material 142. In some embodiments, physical vapor deposition is advantageously used. The conductive layer 146 may be formed of any suitable conductive material, which is preferably also biocompatible, such as, without limitation, niobium, silver, titanium, etc. Selection of the material of the conductive layer 146 may be influenced by the desired sensing function (e.g., impedance measuring capacity), associated circuitry, electrical requirements, signal output requirements, etc.

In order to transmit information sensed by the one or more sensors 140 to the medical professional navigating the leaflet clip delivery and deployment system 100, and/or to transmit power to the one or more sensors 140, one or more leads, traces, connection pathways, etc. may be provided. The leads, traces, connection pathways, etc., may connect one or more wires to the sensors 140. Additionally or alternatively, leads, traces, connection pathways, etc., may connect the one or more sensors 140 with one another. It will be appreciated that the leads, traces, connection pathways, etc., may be formed in any manner known to those of ordinary skill in the art, such as physical vapor deposition, conductive ink printing, preformed electrode bonded via adhesive and/or brazing, etc., the present disclosure not being limited in this context, as such features are not critical to the broad principles of the present disclosure. In some embodiments, a solder layer 148 forms the leads, traces, connection pathways, etc. Additionally or alternatively, a solder layer 148 is provided to form solder pads to facilitate electrical connection of one or more wires 150 to the sensors 140, such as illustrated in FIG. 5. The solder layer 148 may be formed of nickel, copper, gold, or other suitable material. In some embodiments, a primary solder pad of 55-45 nickel-copper is provided on the conductive layer 146, and an optional additional layer of gold may be provided on the primary solder pad, such as to prevent oxidation of the nickel-copper material, such as in a manner known to those of ordinary skill in the art. The solder layer 148 forming one or more of the leads, traces, connection pathways, solder pads, etc., may be formed in any of a variety of manners such as described above with respect to the nonconductive material 142, the tie layer 144, and the conductive layer 146. As may be appreciated by one of ordinary skill in the art, a tie layer 144 such as described above (e.g., a titanium layer) may be provided to facilitate bonding of the soldering layer 148 with the conductive layer 146.

As illustrated in FIG. 3 and in FIG. 5, at least one lead wire 150 extends from at least one sensor 140 proximally along the leaflet clip delivery and deployment system 100 to a proximal location at which power may be supplied to the lead wire 150 and/or the lead wire 150 may be coupled to an indicator which generates a signal from the one or more sensors 140. As may be appreciated, the indicator may be any known indicator configured to provide the desired information in a desired format or configuration to the medical professional operating the leaflet clip delivery and deployment system 100. For instance, the signal may simply be a red light indicating insufficient or no contact, or a green light indicating sufficient or complete contact. Further detailed signals, such as to provide more detailed information with regard to the position, location, degree of purchase or engagement, etc., of the implantable device (e.g., leaflet clip 1000) may be generated in accordance with various principles of the present disclosures, the details of which are not critical to the broad principles of the present disclosure. One or more sensors 140 may be operatively coupled together with leads, traces, connection pathways, etc., so that a single lead wire 150 may be used to supply power to or to transmit a signal from the sensors 140. Additionally or alternatively, each sensor 140 may have its own lead wire(s) 150. The one or more lead wires 150 may be operatively coupled with the one or more sensors 140 via the above-described soldering layer 148, such as in the form of a solder pad. As illustrated in FIG. 3 and FIG. 5, a lead wire 150 may extend from a top of a solder pad formed by a soldering layer 148 on a top surface of an atrial clip arm 1014 of the clip spreader 110 and into a passage 117 in the clip spreader 110 to extend into the clip spreader shaft 106 to extend proximally to a station (such as for power, or a signal indicator device). In some embodiments, the lead wire 150 extends proximally with (e.g., alongside) the above-described actuator 130, such as through the clip spreader shaft 106 and/or other tubular elements (e.g., the delivery catheter 102 or the delivery guide sheath 104) extending proximally to a location outside the patient's body. The clip spreader 110 may be formed with grooves for one or more of the leads, traces, connection pathways, solder pads, lead wires, etc. The lead wire 150 may be potted, such as with a potting material (e.g., epoxy), such as at the entrance to the passage 117, to secure the lead wire 150, such as to inhibit or prevent the lead wire 150 from catching on another object.

As noted above, any of the layers of the one or more sensors 140 and/or any of the leads, traces, connection pathways, solder pads, etc., may be formed in any desired manner including, without limitation, adhesion, interference fit (e.g., fitting in a groove, pocket, recess, etc.), physical vapor deposition (e.g., sputter-coating, thermal evaporation, arc spraying, etc.), electroless plating, electrolytic plating, brazing, or other bonding method. It will be appreciated with respect to any of the above layers of the sensors 140, and/or any of the leads, traces, connection pathways, solder pads, etc., that regions of the leaflet clip delivery and deployment system 100 may be masked or otherwise protected to limit the area to which the material of the sensors 140 and/or any of the leads, traces, connection pathways, solder pads, etc., are applied.

As may be appreciated by one of ordinary skill in the art, it may be challenging to catch a flailing leaflet between the leaflet clip arms 1012, 1014 as the clip spreader 110 opens the leaflet clip 1000 and is moved proximally to catch the heart valve leaflet L between the leaflet clip arms 1012, 1014 (such as illustrated in FIG. 4B). The provision of one or more sensors 140 as disclosed herein allows for confirmation of the desired engagement, placement, purchase, etc., of tissue between the leaflet clip arms 1012, 1014 as the leaflet clip delivery and deployment system 100 (particularly, the clip spreader 110) approaches the heart valve leaflet L. Signals generated by the sensors 140 advantageously facilitate accurate and efficient placement of a leaflet clip 1000 on the heart valve leaflet L.

Although embodiments of the present disclosure may be described with specific reference to a leaflet clip delivery and deployment system 100, it will be appreciated that various other implants may similarly benefit from the devices, systems, and method described herein. For example, other implants for use with mitral valves, or implants which must withstand the palpatory forces for repairing a tricuspid valve annulus and/or addressing other dilatation, valve incompetency, valve leakage, and other similar heart failure conditions may also benefit from the concepts disclosed herein. Furthermore, it will be appreciated that the broad principles and concepts described above with respect to the heart are applicable to other delivery/deployment systems for other types of devices delivered and/or deployed to other locations within a body.

It should be understood that, as described herein, an “embodiment” (such as illustrated in the accompanying Figures) may refer to an illustrative representation of an environment or article or component in which a disclosed concept or feature may be provided or embodied, or to the representation of a manner in which just the concept or feature may be provided or embodied. However such illustrated embodiments are to be understood as examples (unless otherwise stated), and other manners of embodying the described concepts or features, such as may be understood by one of ordinary skill in the art upon learning the concepts or features from the present disclosure, are within the scope of the disclosure. In addition, it will be appreciated that while the Figures may show one or more embodiments of concepts or features together in a single embodiment of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one embodiment can be used separately, or with one or more other features to yield a still further embodiment. Thus, it is intended that the present subject matter covers such modifications and variations as come within the scope of the appended claims and their equivalents.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A delivery/deployment system for transluminally delivering and/or deploying an implantable device at a deployment site, the delivery/deployment system comprising:
a delivery/deployment device configured to deliver and/or deploy an implantable device and including one or more sensors along a portion of the delivery/deployment device configured to engage the implantable device, the one or more sensors being positioned and configured to generate a signal so that the delivery/deployment device and the implantable device need not be imaged to determine the relationship of the implantable device relative to the deployment site.

2. The delivery/deployment system of claim 1, wherein the delivery/deployment device includes a seat in which an implantable device is positionable.

3. The delivery/deployment system of claim 2, wherein the one or more sensors extend along at least one side of the seat.

4. The delivery/deployment system of claim 1, further comprising an indicator coupled with the one or more sensors and configured to generate a signal indicating a condition of the delivery/deployment device and/or an implantable device associated therewith relative to the deployment site.

5. The delivery/deployment system of claim 1, wherein the one or more sensors are formed by a vapor deposition process.

6. The delivery/deployment system of claim 1, wherein the one or more sensors are impedance sensors capable of distinguishing body tissue.

7. The delivery/deployment system of claim 1, wherein the delivery/deployment device is a clip spreader configured to operatively engage a clip having a first clip arm and a second clip arm to shift the clip between an open configuration in which the first clip arm and the second clip arm are spaced apart from each other to allow tissue to be positioned therebetween, and a closed configuration in which the first clip arm and the second clip arm are adjacent to each other to clamp tissue therebetween.

8. A system for delivering and/or deploying an implantable device to a deployment site in a body, the system comprising:
a clip having a first clip arm and a second clip arm, the clip being movable between a closed configuration in which the first clip arm and the second clip arm are adjacent to each other, and an open configuration in which the first clip arm and the second clip arm are spaced apart from each other to allow tissue to be positioned therebetween;
a clip spreader configured to operatively engage the clip to shift the clip between the open configuration and the closed configuration; and
one or more sensors associated with the clip to generate a signal indicating a condition of the clip relative to the deployment site.

9. The system of claim 8, wherein the one or more sensors are provided on the clip spreader to be positioned adjacent the clip engaged by the clip spreader.

10. The system of claim 9, wherein the clip spreader includes a seat in which one of the arms of the clip is engaged, the one or more sensors being arranged along at least one side of the seat to extend along at least one side of the one of the arms of the clip engaged within the seat.

11. The system of claim 10, wherein:
the clip spreader comprises a first spreader arm configured to operatively engage the first clip arm, and a second spreader arm configured to operatively engage the second clip arm;
the seat is formed in at least the second spreader arm;
movement of the first spreader arm away from the second spreader arm moves the clip to the open configuration; and
the one or more sensors are arranged along at least the second spreader arm along at least one side of the seat formed therein.

12. The system of claim 8, wherein the signal indicates at least one or more of the following: purchase of the clip with tissue at the deployment site, level of purchase of the clip with tissue at the deployment site, the position of the clip relative to the deployment site, seating of tissue at the deployment site between the arms of the clip, extent of contact of the clip with tissue at the deployment site, or further information about the clip and/or the clip spreader.

13. The system of claim 8, wherein the one or more sensors generate an audible or visual signal indicating a condition of the clip relative to the deployment site.

14. The system of claim 8, wherein the one or more sensors are formed by a vapor deposition process.

15. The system of claim 8, wherein the one or more sensors are impedance sensors capable of distinguishing body tissue.

16. A method of delivering and/or deploying an implantable device to a deployment site in a body, the method comprising:
delivering an implantable device to a deployment site using a delivery/deployment device, wherein one or more sensors are associated with the implantable device; and
moving the one or more sensors towards the deployment site to cause the one or more sensors to generate a signal indicating a condition of the implantable device relative to the deployment site.

17. The method of claim 16, further comprising deploying the implantable device based on the signal generated by the one or more sensors verifying purchase with tissue.

18. The method of claim 16, wherein the one or more sensors generate a signal upon contact with tissue at the deployment site.

19. The method of claim 16, wherein the implantable device is a leaflet clip having a first clip arm and a second clip arm, the method further comprising:
moving the leaflet clip into an open configuration in which the first clip arm and the second clip arm are spaced apart from each other to allow a heart valve leaflet to be positioned therebetween;
moving the first clip arm and the second clip arm towards each other based on the signal generated by the one or more sensors verifying purchase with tissue.

20. The method of claim 16, wherein the implantable device is a leaflet clip having a first clip arm and a second clip arm, the method further comprising:
moving the leaflet clip into an open configuration in which the first clip arm and the second clip arm are spaced apart from each other to allow a heart valve leaflet to be positioned therebetween;
moving the first clip arm and the second clip arm towards each other and verifying the desired position of the leaflet clip with respect to the heart valve has been reached; and
causing the leaflet clip to clamp onto the heart valve leaflet positioned between the first clip arm and the second clip arm.
